(19) United States
(12) Patent Application Publication
(10) Pub. No.: US 2008/0039666 A1
(43) Pub. Date: Feb. 14, 2008

(54) Composition Comprising Alcohol Alkoxylates And Their Use

(76) Inventors: Rolf Grothe, Marl (DE); Udo Schoenkaes, Haltern (DE); Cronje Grove, Johannesburg (ZA); Ulrich Mayer, Herne (DE); Francesco Monterisi, Milano (IT)

Correspondence Address: C. JAMES BUSHMAN, 5718 WESTHEIMER, SUITE 1800, HOUSTON, TX, 77057, US

(21) Appl. No.: 10/591,136

(22) PCT Filed: Mar. 8, 2004
(86) PCT No.: PCT/EP04/02367

§ 371 Date: Sep. 4, 2007

(51) Int. Cl. C07C 31/00; C11D 1/722

(52) U.S. Cl. 568/840

(57) ABSTRACT

The present invention relates to low foaming non-ionic surfactants being alcohol alkoxylates comprising at least two different oxyalkylene-groups derivable from a mixture of linear an branched alcohols and their use

The present invention relates to low foaming non-ionic surfactants being alcohol alkoxylates comprising at least two different oxyalkylene-groups derivable from a mixture of linear and branched alcohols by alkoxylation and their use.

Non-ionic surfactants are widely used in commercial and household applications where advantage is taken of their valuable performance as wetting agents, their detergency and scouring characteristics, their adaptability for being combined with other types of surfactants and their resistance to hard water conditions. Many of those nonionic surfactants foam too much for applications the extend of foaming is critical, for example, in automatic dishwashers.

In recent years a number of non-ionic low-foaming surfactants have been developed and used commercially. When the need for foam suppression is of prime importance, the non-ionic surfactants developed to meet this requirement often have been found to sacrifice other desirable characteristics, such as detergency, wetting properties, and biodegradation performance.

Non-ionic surfactants based on ethylene oxide and/or propylene oxide adducts are well known in the art. The known surfactants based on ethoxylates and/or propoxylates of detergent range alcohols often show a number of disadvantages, such as that for many applications the foaming behaviour is in need of improvement.

It has long been known to prepare low-foaming non-ionic surfactants by addition of ethylene oxide and/or propylene oxide to various alcohols, generally long chain monohydric alcohols. Numerous different adducts comprising one or more than one alkoxylate in block and/or random structure have been suggested.

Representative prior art relating to alcohol alkoxylates comprising alkoxylates having block structure are described for example in U.S. Pat. No. 4,410,447, U.S. Pat. No. 3,956,401 and EP 0 882 785-A1.

EP 0 882 785-A1 describes a non-ionic surfactant which has low foaming power, detergency characteristics, and consists of a product having the general formula (I):

RO—(EO)x—(PO)y—(EO)z—H (I)

wherein

R represents a linear or branched C12 to C15 alkyl chain,

EO and PO are oxyethylene and oxypropylene units respectively, and

x and z are stoichiometric indices and are equal to or higher than 1 and

y represents a mean number ranging from 0.5 to 6.

The surfactant having general formula (I) however, has a series of limitations, such as that the molecular weight of this product, must not be higher than 1200 otherwise the detergent properties deteriorate or the surfactant cannot be diluted in water in quantities of less than 50% by weight as with lower concentrations there are significant variations in viscosity. The succession of EO and PO indexes must remain within certain limits to avoid a reduction in detergent properties. These restrictions, and others specified in the test procedure described in EP 0 882 785-A1, make the surfactant having general formula (I) of little interest from an industrial point of view.

U.S. Pat. No. 3,956,401 describes a low foaming, biodegradable non-ionic surfactant which is liquid at room temperature and non-gelling in water solution. The surfactant has the general formula (II):

RO—(CH₂CHR′—O)ₓ—(CH₂—CH₂—O)_{y}—(CH₂—CHR″—O)_{z}—H (II)

wherein R is a linear alkyl group containing 7 to 10 carbon atoms,

R′ and R″ are most desirably a methyl group,

x is an integer of 1 to 6,

y is an integer of 4 to 15 and

z is an integer of 4 to 25.

This detergent comprises successively an oxypropylene (PO) block, an oxyethylene (EO) block and an oxypropylene (PO) block bound to the alcohol.

U.S. Pat. No. 4,410,447 also describes a liquid low-foaming non-ionic surfactant, which is also said to have good scouring and/or detergency. This surfactant has the formula (III):

R—O—Ax—B—H (III)

wherein

R is a primary alkyl group having from 7 to 11 carbon atoms,

A is a oxypropylene group, with the proviso that the total number of carbon atoms in R plus A is 12 to 22,

x is an integer from 2 to 15, and

B is a random mixture of oxyethylene and oxypropylene groups, wherein the molar ratio of oxyethylene to oxypropylene in A and B is from 0.2:1 to 1.5:1 while the molar ratio of oxyethylene to oxypropylene in B is from 1:1 to 5:1.

This prior art patent, therefore, discloses a detergent which comprises successively a PO block adducted to the alcohol and a random mix of EO and PO units. Respective compositions have a cloud point from about 20 to about 60° C.

Common to all prior art patents is the method to use alcohols having a particular number of carbon atoms in relation to certain amounts of ethylene oxide and/or propylene oxide to obtain products that are liquid and low foaming and have superior wetting and detergency properties. An alternative approach in the patent literature to solve above problem is by modifying the hydrophobe moiety of the surfactant molecule. Such an approach is for example described in U.S. Pat. No. 6,057,284. U.S. Pat. No. 6,057,284 describes a low-foaming non-ionic surfactant which is using isotridecanol as the hydrophobe moiety of the surfactant and has the formula (IV):

RO—(CₘH₂ₘO)x—(CₙHₙ—O)y—H (IV)

wherein

R is isotridecyl which is mainly based on primary C13 alkanols having at least 3 branches,

m is 2 when n is 3 or 4, or

m is 3 or 4 when n is 2, and

x and y are independently of each other from 1 to 20, with the proviso that

x is not less than y when m=2, and n=3 or 4.

The isotridecanol (isotridecyl alcohol) is of synthetic origin in that it is produced by oligomerization of suitable lower olefins and subsequent oxo reaction (hydroformylation). For instance, isobutylene, 1-butylene, 2-butylene or mixtures thereof can be catalytically trimerized, propylene catalytically tetramerized or 2-methyl-1-pentene catalytically dimerized. The C12 olefins thus obtainable are then converted to the homologous mologous C13 alcohol, for example by means of CO addition and hydration over a suitable catalyst.

However, the underlying alcohol component need not be just pure isotridecanol; homologous mixtures of branched C11 to C14 alkanols comprising isotridecanol as main component are also suitable. Such homologous mixtures are formed under certain conditions in the course of the above-described oligomerisation of lower olefins and subsequent oxo reaction.

The degrees of alkoxylation x and y, which in general are averages, since the alkylene oxide units are usually present in a random distribution with a frequency maximum, are preferably independently of each other from 1.5 to 12. The disclosed isotridecanol block alkoxylates are either ethylene oxide-propylene oxide or ethylene oxide-butylene oxide adducts.

However, known surfactants based on isotridecanol alkoxylates in the field of detergents and cleaners and in the field of chemical-technical applications have a number of disadvantages. Particularly physical and application properties such as surface tension, cleaning power and foam behavior are in need of improvement. Due to the multibranched structure of the alcohol hydrophobe the PO containing isotridecanol adducts often have only unsatisfactory biodegradation properties.

Alcohol(C1 to C6)alkoxylates of Fischer Tropsch (FT) derived alcohols are for instance known from DE 20303420-U1 (page 4). Furthermore in EP 0 329 670-B1 (page 23) semi-linear surfactants derived from ethoxylated and/or propoxylated alcohols are taught. Semi-linear alcohol alkoxylates are as well disclosed in some of the Procter & Gamble applications directed to mid-chain branched alcohol derivatives of which EP 0 898 607-B1(=WO 97/39089) is an example. However, none of above cited references disclose the specific structure, composition and use of the subject matter of this invention.

Biodegradation is a crucial property of alcohol alkoxylates comprising propylene oxide units, because the incorporation of propylene oxide negatively influences the biodegradability of the molecule. In the technical literature it is described that known alcohol-EO-PO adducts often have only poor biodegradation properties, which often prevents their widespread use. In the journal Tenside Detergents 23(1986),6, p. 300-304 Gerike and W. Jasiak state that for many alcohol derivatives biodegradability is often impaired by propoxylation, e.g. in the Coupled Units Test a C12 to C14 fatty alcohol with 4EO units and 5 PO units showed only a C-removal of 49%.

In Tenside Detergents March/April 1988, p. 86-107 a C12-C18 fatty alcohol alkoxylated with 2.5 EO and 6 PO units is reported to have only a biodegradation of 37% (Coupled Units Test). The compound did not reach the pass level for ready biodegradability.

As the detergent markets move to more environmental safe products, and more stringent environmental regulations are coming-up, e.g. the new EU Detergent Directive, there is need for low-foaming detergents that offer an improved biodegradation performance, along with good physico-chemical properties and good cleaning abilities.

The known low-foam surfactants do not have a suitable combination of properties of foam suppression, wetting action, biodegradation performance and ability to be formulated which is suitable for all applications.

With the objective of finding a non-ionic surfactant with a low foaming power which does not have the disadvantages of those of the known art it was now surprisingly discovered that by using an alcohol derivable from the hydroformylation of a Fischer-Tropsch olefin, a non-ionic surfactant can be obtained which satisfies the required requisites, that impairs excellent physico-chemical and detergent properties and superior biodegradability with respect to traditional low-foaming surfactants.

The non-ionic surfactant with low foaming power comprises a mixture of alcohol alkoxylates that have the general formula (V):

RO—[(CH₂CHR¹O)ₓ(CH₂CHR²O)_{y}]_{z}—H (V).

The feedstock alcohols R-OH are alcohols that can be prepared by the hydroformylation of olefins that are obtained in the FT process from synthesis gas.

The feedstock alcohols used are a mixture of alcohols ROH, being essentially primary alcohols, consisting of

(a) from more than 20 to 80% by mass, preferably 40 to 80% by mass, of alcohols that are linear and aliphatic, preferably saturated, and comprise 8 to 20, preferably 9 to 16, and more preferably 10 to 14 carbon atoms,

(b) from more than 10 to 80% by mass, preferably 40 to 80% by mass, of alcohols that are aliphatic, preferably saturated, and comprise

8 to 20, preferably 9 to 16 and more preferably 10 to 14 carbon atoms, and

up to 3, preferably 1 or 2, carbon atoms are tertiary carbon atoms whereas

none of the two carbon atoms in the 1 or 2 position, preferably none of the three carbon atoms in the 1, 2, or 3 position, relative to the OH group, is a tertiary carbon atom,

preferably none of the two, preferably none of the three carbon atoms at the end of the chain are tertiary carbon atoms, and

(c) up to 25% by mass, preferably 10 to 20% by mass, of alcohols different to (a) and (b) comprising 8 to 20, preferably 9 to 16, and more preferably 10 to 14 carbon atoms,

wherein for all alcohols according to (a), (b) and (c)

at least 80%, preferably at least 95%, of the tertiary carbon atoms, related to the total of all tertiary carbon atoms in the alcohol mixture, are not directly adjacent,

the alcohols according to (a), (b) and (c) supplement one another essentially to 100% by mass and

wherein for the alcohols (b) and (c) that may comprise alkyl branching

at least 80%, preferably at least 95%, of the alkyl branches are methyl and/or ethyl.

Such alcohol mixtures are available on the market and sold by SASOL Ltd, South Africa as SAFOL™ 23, SAFOL™ 25, and SAFOL™ 45. These alcohols are derived from the hydroformylation of C11 to C12 olefins, C13 to C14 olefins and their mixtures respectively obtained from high temperature Fischer-Tropsch reaction.

Most preferably a C12 to C13 Fischer-Tropsch alcohol (SAFOL™ 23 alcohol) is used. SAFOL 23 is a primary alcohol and consists of ca. 50% by mass linear alcohol isomers, ca. 30% mono-methyl branched alcohol isomers and ca. 20% by mass by mass of other isomeric alcohols.

### R¹, R², x, y and z in general formula V:

RO—[(CH₂CHR¹O)ₓ(CH₂CHR²O)_{y}]_{z}—H (V).

are defined as follows:
R¹ and R² are independent of one another and optionally different for each z, selected from the group consisting of: H and linear aliphatic C1 to C3 hydrocarbons and preferably are methyl and/or ethyl with the proviso that R¹ and R² are not the same for one z,

x and y are independent of one another and optionally different for each z values from 1 to 10, preferably with the proviso that at least one x or y is equal to or greater than 2 and more preferably 2 to 10, and

z has a value of from 1 to 5.

Further wherein R¹ is H, x preferably falls in the range from 1 to 10, more preferably in the range 1 to 6, with R methyl, ethyl or propyl and y preferably in the range from 1 to 10, more preferably in the range 1 to 6. z is preferably in the range 1 to 2, more preferably 1.

Alternatively when R¹ is methyl, ethyl or propyl, x preferably falls in the range from 1 to 10, more preferably in the range 2 to 6, with R² equals H and y preferably in the range from 1 to 10, more preferably in the range 2 to 6. z is preferably in the range 1 to 2, more preferably 1.

The non-ionic surfactant having the general formula (V) can be prepared with known techniques, for example by reacting an alcohol R—OH with ethylene oxide and propyl-ene oxide or butylene oxide, alternating blocks of the former with blocks of the latter, in the presence of a base catalyst selected from the hydroxides of alkaline or earth-alkaline metals or from mixed oxides of magnesium-zinc, magnesium-tin, magnesium-titanium or magnesium-antimony, or acids like H₂SO₄, or Lewis acids like TiCl₄. Also catalysts based on a mixture of calcium hydroxide, dispersed in an alcohol ethoxylate medium, partially neutralized with 2-ethylhexanoic acid and sulfuric acid and also catalysts based on a mixture of calcium hydroxide, dispersed in an alcohol ethoxylate medium, partially neutralized with 2-phenylhexanoic acid and sulfuric acid and mixed with aluminium alkoxide, can be used. More preferred are the catalysts KOH, NaOH, and MeONa. The catalysts are preferably employed at amounts of about 0.1 mol % to about 3.0 mol %, with 0.2 mol % to about 1.0 mol % being more preferred.

The Fischer-Tropsch (FT) process is a process for the synthesis of hydrocarbons. Synthesis gas, a mixture of hydrogen and carbon monoxide, is reacted in the presence of an iron or cobalt catalyst. An important source of the hydrogen-carbon monoxide gas mixture is the gasification of coal. The process is named after F. Fischer and H. Tropsch, the German coal researchers who discovered it in 1923.

In the Fischer-Tropsch process, synthesis gas (carbon monoxide and hydrogen) obtained from gasification of coal or reforming of natural gas, is reacted over a Fischer Tropsch catalyst to produce a mixture of hydrocarbons ranging from methane to waxes and smaller amounts of oxygenates.

In a low temperature Fischer-Tropsch reaction, the reaction takes place in a slurry bed reactor or fixed bed reactor, preferably a slurry bed reactor, at a temperature in the range of 160° C. to 280° C., preferably 210° C. to 260° C., and a pressure in the range of 18 to 50 bar, preferably between 20 to 30 bar, in the presence of a catalyst. The catalyst may include iron, cobalt, nickel or ruthenium. However, a cobalt-based catalyst is preferred for the low temperature reaction. Usually, the cobalt catalyst is supported on an alumina support. The hydrocarbon condensation product includes olefins and paraffins in the C4 to C26 range, and oxygenates including alcohols, esters, aldehydes, ketones, and acids.

In a high temperature Fischer-Tropsch reaction, the reaction takes place in a two phase reactor containing gas and catalyst, preferably a fluidized bed reactor, at a temperature range of 320° C. to 350° C., preferably 330° C. to 350° C., and a pressure in the range of 18 to 50 bar, preferably between 20 to 30 bar. The catalyst comprises iron promoted with alkali, prepared in existing commercial applications from fused magnetite. The iron oxide catalyst precursor is at least partially reduced to the metal prior to loading it into the synthesis reactor and it typically is subjected to reactions and phase changes during operation at synthesis conditions that change the catalyst structure and composition.

The products typically comprises linear and branched olefin products.

Particulars of the process useful for the production of hydrocarbons that may be used as starting material to obtain the desired alcohol mixture of the present invention are as well described in Dry, M E, in “Catalysis-Science and Technology”, Anderson, J R and Boudart, M (eds.), Springer-Verlag, Berlin, 159 (1981).

### Use and Application

The alcohol alkoxylates of the present invention can be used as low-foaming, foam-suppressing, and anti-foam surfactants. They are suitable, in particular, for detergents and cleaners, and for industrial and chemical-technical applications. Low-foaming refers to the fact that respective compounds have a lower tendency to form foam compared to equivalent compounds having the same structure with respect to the alcohol group and the same number of alkoxy groups but being alkoxylated with ethylenoxide only.

The disclosed FT alcohol alkoxylates can be applied in a wide range of household and industrial and institutional (I&I) cleaning formulations. Examples of typical applications include household and I&I laundry detergents, household and I&I machine dish wash, household and I&I rinse aids, household and I&I hard surface cleaning, car cleaners, bottle washing, metal cleaning, spray cleaning, machine cleaning (e.g. floor cleaning machines), and cleaners for breweries and butcheries.

The present invention likewise provides detergent and cleaning formulations which, as well as the customary constituents, contain the alcohol alkoxylates of formula (V) in concentrations of up to 50%, preferably from 0.1 to 20%, more preferably 0.1 to 15% by weight.

Besides, in the field of detergents and cleaners the claimed alcohol alkoxylates can be used in textile processing, leather processing, metal processing, cooling lubricants, water treatment, fermentation, paper processing, latex paints, crop protection formulations, and industrial processing.

In addition to the alkoxylates based on FT alcohols the compositions and formulations according to the present invention may contain also other anionic, nonionic and cationic surfactants, or mixtures thereof. Customary surfactants that can be used with the FT alcohol alkoxylates of this invention are, for instance, described by Kurt Kosswig in Ullmann's Encyclopedia of Industrial Chemistry, Vol. A25 (1994), p. 747-817.

Suitable anionic surfactants that can be present include sulfonates and sulphates. Examples of sulfonate-type surfactants include linear alkylbenzene sulfonates, e.g. C10- to C13- LAS, C13- to C17-paraffin sulfonates, and ester sulfonates having chain lengths of 12 to 20 C atoms. Examples of sulfate-type surfactants include the monoesters of sulfuric acid with fatty alcohols of synthetic and natural origin, such as coconut fatty alcohol, tallow fatty alcohol, oleyl alcohol, or C10 to C20 oxo alcohols. Fatty alcohol ether sulfates, such as lauryl ether sulfate, C12 to C13 alcohol ether sulfates, C12 to C15 alcohol ether sulfates, or butylglycol sulfate, may also be used.

Other suitable anionic surfactants are soaps, including saturated fatty acid soaps, such as the alkali metal or alkanolamine soaps of lauric acid, myristic acid, palmitic acid and stearic acid. Soap mixtures derived from natural fatty acids, such as coconut, palm kernel or tallow fatty acids, are preferred.

Suitable nonionic surfactants include adducts of ethylene oxide and/or propylene oxide with alkylphenols, oxo alcohols or natural or Ziegler alcohols, fatty acids, fatty amines and fatty acid amides. The adducts of 3 to 15 mol of ethylene oxide with coconut and tallow fatty alcohols, with oleyl alcohol or with synthetic alcohols having 8 to 18 C atoms are particularly preferred. Also alcohol-EO-POs already on the market, as they are sold under the trade names Biodac® and Marlox® (Sasol), Plurafac® LF (BASF) and Dehypon® LS (Cognis), and other suppliers can be used. Besides this also high-ethoxylated tallow fatty alcohols or fatty alcohol ethoxylates that are end-capped by alkyl groups can be used as additional wetting agents. Surfactants of the type comprising the C8 to C18-alkylpolyglucosides and amine oxides may also be used.

It is furthermore possible to use cationic surfactants and amphoteric products, such as ampholytes and betaines.

### EXAMPLES

For illustrative and non-limiting purposes, non-ionic surfactants having the general formula (V) were prepared wherein R is a mixture of C12 to C13 linear and branched alkyl with R₁=H and R₂=CH₃ and CH₂ CH₃ and the index x was between 1 and 10, and y between 1 and 6 with z=1.

RO—[(CH₂CHR₁O)ₓ(CH₂CHR₂O)_{y}]_{z}—H (V)

297 grams of C12 to C13 FT alcohol (SAFOL™ 23 alcohol) were placed in an auto-clave into which 3.9 grams of potassium hydroxide (50% in water) were charged. The autoclave was then heated to 150° C. under vacuum to dehydrate the reaction mixture. The temperature was then brought to 160° C., and 330 grams of ethylene oxide were slowly fed. At the end of the reaction of the ethylene oxide, when the pressure drop had been stabilized, 350 grams of propylene oxide were fed. At the end of the reaction the product was cooled to 80° C. and neutralized up to pH 6 with acetic acid.

Physical-chemical characteristics of alcohol-EO-POs of examples of the present invention prepared according to the above mentioned method are given in Table 1.

TABLE 1

| | | Cloud | Wetting | Foaming |
|---|---|---|---|---|
| | Appearance | point | power | power |
| Product | at 20° C. | [° C.] | [sec] | [cm] |
| C12-C13 FT alcohol + | Liquid | 41 | >200 | 1 |
| 2EO + 2PO | | | | |
| C12-C13 FT alcohol + | Liquid | 39 | 100 | 1 |
| 2EO + 4PO | | | | |
| C12-C13 FT alcohol + | Liquid | 48 | 15 | 3 |
| 5EO + 4PO | | | | |
| C12-C13 FT alcohol + | Liquid | 40 | 15 | 2 |
| 5EO + 5PO | | | | |
| C12-C13 FT alcohol + | Liquid | 35 | 15 | 1 |
| 5EO + 6PO | | | | |

The cloud point was determined in butyl diglycol by using the method DIN EN 1890. This involved determining the temperature above which the solution turns cloudy. The lower the cloud temperature, the lower the foaming tendency.

The wetting power was determined at 20° C. by method DIN EN 1772. The wetting effect corresponds to the time taken for a cotton disk to sink in aqueous solution. The shorter the sinking time, the better the wetting efficiency.

The foaming power was determined at 40° C. under high pressure conditions with a 1 g/l active surfactant solution in a foam-test cabin. The test apparatus consists of a reservoir for the surfactant solution, a pump and a test cabin. The foam is generated by spraying the surfactant solution with pressure onto a wire netting (placed in the cabin) and the foam height in the cabin is determined by a graduation (in cm). In this test low-foaming alcohol-EO-POs from the market typically showed foam heights of 1 to 10 cm, regular alcohol ethoxylates with 7 moles EO produce foam heights of more than 20 cm.

In the following properties of the non-ionic surfactants of the present invention are compared to typical traditional surfactants based on a linear alcohol (Table 2).

TABLE 2

| | Wetting | |
|---|---|---|
| Product | power [sec] | Foaming power [cm] |
| C12-C13 FT alcohol-5EO-4PO | 15 | 3 |
| Linear C12-C14 alcohol-5EO-4PO | 20 | 3 |

Compared to the traditional linear C12 to C14 alcohol-5EO-4PO from the market the C12-C13-FT alcohol-5EO-4PO of the present invention had a better wetting performance.

Biodegradability tests carried out on C12 to C13 FT alcohol-2EO-4PO, C12-C13 FT alcohol-5EO-4PO and C12-C13 FT alcohol-5EO-6PO gave excellent results with respect to the total biodegradability, and the given products turned out to be readily biodegradable.

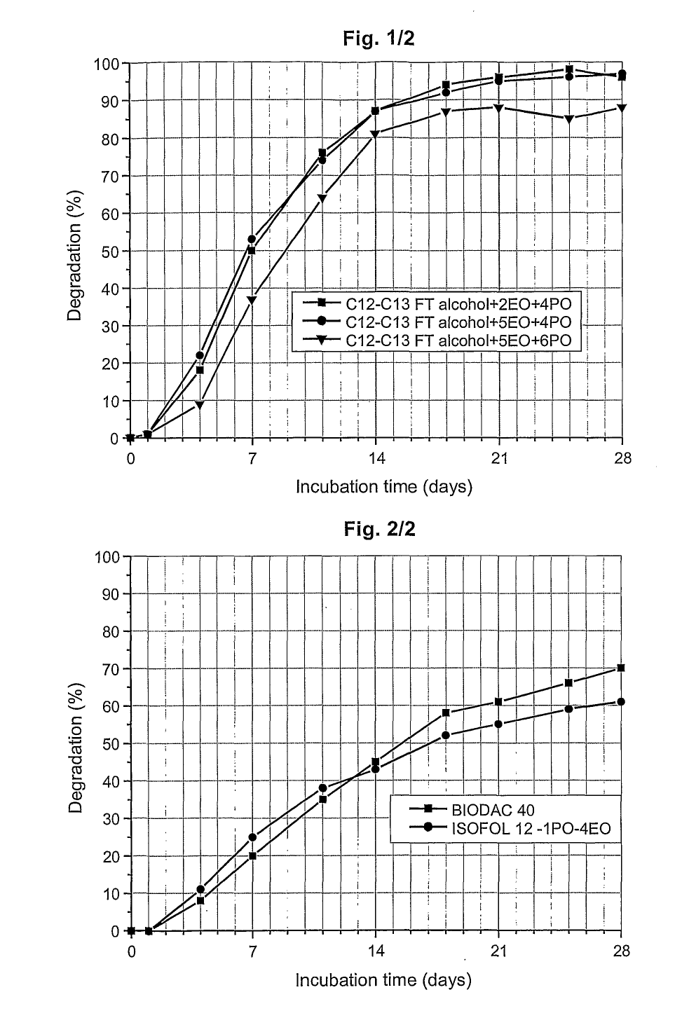

FIG. 1 shows the biodegradation performance of C12-C13-FT alcohol-EO-POs (CO₂ Evolution Test on “ready” biodegradability according to OECD 301 B).

All three tested FT alcohol-EO-POs reached the pass level of 60% for ready biodegradability within the 10-day window:

C12-C13 FT alcohol+2EO+4PO reached 83% at the end of the 10-day window

C12-C13 FT alcohol+5EO+4PO reached 79% at the end of the 10-day window

C12-C13 FT alcohol+5EO+6PO reached 81% at the end of the 10-day window.

Therefore all three tested products can be termed “readily” biodegradable.

The biodegradation of the FT alcohol-EO-PO derivatives was compared with traditional alcohol-EO-POs (FIG. 2, CO₂ Evolution Test on “ready” biodegradation according to OECD 301 B).

Biodac® 40 is an alcohol EO-PO based on a linear Ziegler-alcohol, with an alkyl chain having 10 carbon atoms, ethoxylated with 4 moles of ethylene oxide and propoxylated with 2 mole of propylene oxide.

Isofol® 12-1PO-4EO is based on a branched Guerbet alcohol, with an allyl chain having 12 carbon atoms, propoxylated with 1 mole propylene oxide and then ethoxylated with 4 moles of ethylene oxide.

Compared to the C12-C13 FT alcohol-EO-POs of the present invention Biodac 40 and Isofol® 12-1PO-4EO showed a lower biodegradation level at the end of the 28 day period. Both Biodac® 40 and Isofol 12-1PO-4EO did not reach the 60% level for “ready” biodegradation:

Biodac® 40 reached 48% at the end of the 10-day window

Isofol® 12-1PO-4EO reached 42% at the end of the 10-day window.

Therefore both products do not fulfill the 10-day window criterium and are not classified as “ready” biodegradable.

Typical formulation examples to use the alcohol alkoxylates of this invention in machine dish wash powders and in liquid rinse aids with low-foaming characteristics are given in Table 3 (Dish washing detergents) and Table 4 (Rinse aids).

The given formulations showed a low-foaming characteristic when applied in dish-washing machines and a good cleaning and rinsing performance.

TABLE 3
Automatic Dishwashing Powders

| Example A (phosphate-free powder) | |
|---|---|
| Sodium disilicate | 25% |
| Sodium citrate dihydrate | 35% |
| C12-13 FT alcohol-5EO-4PO | 2.5% |
| Polycarboxylate | 5% |
| Sodium perborate mono hydrate | 15% |
| Tetraacetylethylenediamine (TAED) | 4% |
| Enzymes | 4% |
| Sodium sesquicarbonate | to 100 |
| Example B (powder based on phosphate) | |
| C12-C13-FT alcohol-5EO-4PO | 1% |
| Sodium tripolyphosphate | 40% |
| Sodium metasilicate | 35% |
| Sodium carbonate | 22% |
| Sodium dichlor isocyanurate | 2% |

TABLE 4
Rinse aids for machine dishwash

| | |
|---|---|
| Example C | |
| C12-C13 FT alcohol-5EO-4PO | 10% |
| C16-C18 fatty alcohol-25EO | 5% |
| (MARLIPAL ® 1618/25, Sasol Germany) | |
| Citric acid | 3% |
| Isopropanol | 15% |
| Water | to 100 |
| Example D | |
| C12-C13 FT alcohol-5EO-4PO | 10% |
| Sodium cumene sulfonate*, 40% | 10% |
| Citric acid | 5% |
| Water | to 100 |

*(Na Cumene sulfonate 40, Sasol Germany)

1. Composition comprising alcohol alkoxylates of the general formula (V)
RO[(CH₂CHR¹0)ₓ(CH₂CHR²0)_{y}]_{z}—H (V)
wherein the residue RO is derivable from a mixture of alcohols ROH, being essentially primary alcohols, essentially consisting of
(a) from 20 to 80% by mass of alcohols that are linear and aliphatic and comprise 8 to 20 carbon atoms,
(b) from 10 to 80% by mass of alcohols that are aliphatic and comprise
8 to 20 carbon atoms, and
1, 2 or 3 carbon atoms are tertiary carbon atoms whereas
none of the two carbon atoms in the 1 or 2 position relative to the
OH group is a tertiary carbon atom and
(c) up to 25% by mass of alcohols different from (a) and (b) and comprise 8 to 20 carbon atoms,
wherein for all alcohols according to (a), (b) and (c)
at least 80% of the tertiary carbon atoms related to the total of all tertiary carbon atoms in the alcohol mixture are not directly adjacent,
the alcohols according to (a), (b) and (c) supplement one another essentially to 100% by mass and
wherein for the alcohols (b) and (c) that have alkyl branching
at least 80% of the alkyl branches are methyl and/or ethyl and
R¹ and R² are independent of one another and optionally different for each z, and are selected from the group consisting of
H and linear aliphatic C1 to C3 hydrocarbons with the proviso that
R¹ and R² are not the same for one z,
x and y are independent of one another and optionally different for each z value from 2 to 10, and
z has a value of from 1 to 5.

2. Composition according to claim 1, wherein
x is from 2 to 6,
y is from 2 to 6,
z is from 1 to 2, preferably 1, and
R¹ is H and R² is methyl, ethyl or propyl or
R¹ is methyl, ethyl or propyl and R² is H.

3. Composition according to any one of claims 1 or 2, wherein the alcohol mixture comprise independently of each other
40 to 80% by mass of alcohol (a),
40 to 80% by mass of alcohol (b) and/or
10 to 20% by mass of alcohol (c).

4. Composition according to any one of claims 1 or 2, wherein alcohols (a), (b) and/or (c) comprise independently from each other 9 to 16 carbon atoms.

5. Composition according to any one of claims 1 or 2, wherein for alcohols (b) 1 or 2 carbon atoms are tertiary carbon atoms and independently none of the three carbon atoms in the 1, 2 or 3 position relative to the OH group is a tertiary carbon atom.

6. Composition according to any one of claims 1 or 2, wherein for alcohols (b) none of the two, carbon atoms at the end of the chain are tertiary carbon atoms.

7. Composition according to any one of claims 1 or 2, wherein at least 95% of the tertiary carbon atoms relative to the total of all tertiary carbon atoms in the alcohol mixture are not directly adjacent.

8. Composition according to any one of claims 1 or 2, wherein at least one x or y is equal to or greater than 2.

9. Composition according to any one of claims 1 or 2, wherein the alcohols ROH are derivable from olefins obtainable by Fischer Tropsch Synthesis via hydroformylation.

10. (canceled)

11. Composition of matter according to claim 19, wherein the composition of matter additionally comprises other anionic, nonionic and/or cationic surfactants, or mixtures thereof.

12. Composition of matter according to claim 11, wherein the additional anionic surfactants are selected from the group consisting of sulfonates and/or sulfates.

13. Composition of matter according to claim 11, wherein the additional nonionic surfactants are selected from the group consisting of adducts of ethylene oxide and/or propylene oxide, of alkylphenols, fatty acids, fatty amines and/or fatty acid amides and/or adducts of ethylene oxide to C8 to C18 fatty alcohols.

14-15. (canceled)

16. The composition of claim 4, wherein said alcohols (a), (b) and/or (c) comprise independently from each other 10 to 14 carbon atoms.

17. The composition of claim 4, wherein said alcohols (a), (b) and/or (c) comprise independently from each other 12 to 13 carbon atoms.

18. The composition of claim 8, wherein x and y are from 2 to 10.

19. Composition of matter comprising up to a concentration of 50% by weight of the composition of any one of claims 1 or 2.

20. Composition of matter according to claim 19, wherein the concentration is from 0.1 to 20% by weight.

21. Composition of matter according to claim 19, wherein the concentration is from 0.2 to 15% by weight.

22. Composition of matter of claim 12, wherein said additional anionic surfactants are selected from the group consisting of C9 to C16 alkyl benzolsulfonates, C12 to C18 alkyl sulfates, C12 to C15 alkyl ether sulfates, butylglycol sulfate and mixtures thereof.

23. Composition of matter of claim 11, wherein said additional anionic surfactant comprises saturated C12 to C18 fatty acid soaps.

24. Composition of matter of claim 13, wherein said adducts of said C8 to C18 fatty alcohols contain from 3 to 15 mols of ethylene oxide.

25. Composition according to any one of claims 1 or 2, wherein for alcohols (b) none of the three, carbon atoms at the end of the chain are tertiary carbon atoms.
